(19) United States
(12) Patent Application Publication
(10) Pub. No.: US 2011/0193068 A1
(43) Pub. Date: Aug. 11, 2011

(54) Composite film, flexible substrate including the composite film, and organic light emitting device including the flexible substrate

(76) Inventors: Jin-O Lim, Yongin-City (KR); Kie-Hyun Nam, Yongin-City (KR)

(21) Appl. No.: 12/926,760

(22) Filed: Dec. 8, 2010

(30) Foreign Application Priority Data
Feb. 11, 2010 (KR) 10-2010-0012895

(51) Int. Cl. H01L 51/30; C09K 11/06; C07C 309/00

(52) U.S. Cl. 257/40; 252/301.35; 562/108; 257/E51.024

(57) ABSTRACT

A composite film, a flexible substrate, and an organic light emitting device, the composite film including a composite including a sulfonic acid group containing moiety connected by an ether linkage (—O—) to an inorganic material having a nano-sized interlayer distance.

### BACKGROUND

1. Field

Embodiments relate to a composite film, a flexible substrate including the composite film, and an organic light emitting device including the flexible substrate.

2. Description of the Related Art

Flexible substrates that are thin, light-weighted, and have excellent impact resistance are increasingly desirable. Highly flexible substrates are widely used in liquid crystal display devices and organic light emitting display devices.

A flexible substrate included in an organic light emitting display device may be formed of an organic material. Thus, if the flexible substrate is exposed to oxygen or moisture, a lifespan of the flexible substrate may be significantly reduced. Accordingly, use of a metal foil and plastic substrate has been studied.

Currently, studies on plastic substrate materials including a polymer film are being widely conducted. Engineering polymer materials having excellent physical properties, e.g., poly(arylene ether sulfone), polycarbonate, and polyimide, are mainly studied as the plastic substrate materials. These polymer materials may have excellent flexibility and an insulation property required in a metal foil. The polymer materials may reduce a high coefficient of thermal expansion (CTE), which is one disadvantage of metals, to a nearly similar level to that of glass through a polymer structure. Thus, accurate patterning is possible when manufacturing a thin film transistor (TFT).

### SUMMARY

Embodiments are directed to a composite film, a flexible substrate including the composite film, and an organic light emitting device including the flexible substrate, which substantially overcome one or more of the problems due to the limitations and disadvantages of the related art.

It is a feature of an embodiment to provide a composite film having an excellent resistance to moisture vapor transmission.

At least one of the above and other features and advantages may be realized by providing a composite film including a composite including a sulfonic acid group containing moiety connected by an ether linkage (—O—) to an inorganic material having a nano-sized interlayer distance.

The sulfonic acid group containing moiety may include —(CH₂)ₙSO₃M, wherein M is Na, K, Ca, or Ba and n is and integer of about 1 to about 13.

The sulfonic acid group containing moiety may include —C(R₂)(X)CF₂SO₃M, wherein M is Na, K, Ca, or Ba, R₂ is —F, —CF₃, —SF;, ═SF₄, —SF₄Cl, —CF₂CF₃, or —H(CF₂)₄, and X is —F, —H, —Cl, or —CF₃.

The inorganic material may include at least one of montmorillonite, koline, hydrated sodium calcium aluminum magnesium silicate hydroxide, pyrophyllite, talc, vermiculite, sauconite, saponite, nontronite, amesite, baileychlore, chamosite, clinochlore, kaemmererite, cookeite, corundophilite, daphnite, delessite, gonyerite, nimite, odinite, orthochamosite, penninite, pannantite, rhipidolite, prochlore, sudoite, thuringite, kaolinite, dickite, and nacrite.

The composite may be obtained by sulfonation of the inorganic material by a sultone compound.

The sultone compound may include at least one of 1,3-propane sultone, 1,4-butane sultone, and 1-trifluoromethyl-1,2,2-trifluoroethane sulfonic acid sultone.

The composite film may further include a polymer.

The polymer may include at least one of polyimide, polyester, polycarbonate, and polyethylene terephthalate.

The composite may be included in an amount of about 0.1 parts by weight to about 10 parts by weight based on 100 parts by weight of the polymer.

At least one of the above and other features and advantages may also be realized by providing a flexible substrate including a composite film including a composite including a sulfonic acid group containing moiety connected by an ether linkage (—O—) to an inorganic material having a nano-sized interlayer distance.

The composite film may further include polyimide.

The sulfonic acid group containing moiety may be —(CH₂)₃SO₃Na.

At least one of the above and other features and advantages may also be realized by providing an organic light emitting device including a first electrode, a second electrode, and an organic film interposed between the first electrode and the second electrode; and a composite film including a sulfonic acid group containing moiety connected by an ether linkage (—O—) to an inorganic material having a nano-sized interlayer distance.

The composite film may be a flexible substrate.

The composite film may be an encapsulation film on the second electrode.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 illustrates a process of manufacturing an inorganic material directly connected to a sulfonic acid group by an ether linkage, according to an embodiment;

FIG. 2 illustrates an organic light emitting device according to an embodiment;

FIG. 3 illustrates an organic light emitting device according to another embodiment; and

FIG. 4 illustrates an organic light emitting device according to yet another embodiment.

### DETAILED DESCRIPTION OF THE INVENTION

Korean Patent Application No. 10-2010-0012895, filed on Feb. 11, 2010, in the Korean Intellectual Property Office, and entitled: “Composite Film, Flexible Substrate Including the Composite Film, and Organic Light Emitting Device Including the Flexible Substrate,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

Hereinafter, a composite film, a flexible substrate including the composite film, and an organic light emitting device including the flexible substrate are described more fully with reference to the accompanying drawings.

According to an embodiment, a composite film may include a composite in which a sulfonic acid group containing moiety is connected to an inorganic material having a nano-sized interlayer distance by an ether linkage.

The sulfonic acid group containing moiety may be a functional group that adsorbs moisture and has a high barrier property against gas and moisture. In an implementation, the sulfonic acid group containing moiety may be, e.g., —(CH₂)ₙSO₃M, (wherein M may be Na, K, Ca, or Ba, and n may be an integer of about 1 to about 13). In another implementation, the sulfonic acid group containing moiety may be, e.g., —C(R₂)(X)CF₂SO₃M (wherein M may be Na, K, Ca, or Ba, R₂ may be —F, —CF₃, —SF_{S}, ═SF₄ (wherein “═” represents a double bond), —SF₄Cl, —CF₂CF₃, or —H(CF₂)₄, and X may be —F, —H, —Cl, or —CF₃.

The inorganic material may have a low coefficient of thermal expansion (CTE) and high thermal stability. In an implementation, the inorganic material may include, e.g., montmorillonite, koline, hydrated sodium calcium aluminum magnesium silicate hydroxide, pyrophyllite, talc, vermiculite, sauconite, saponite, nontronite, amesite, baileychlore, chamosite, clinochlore, kaemmererite, cookeite, corundophilite, daphnite, delessite, gonyerite, nimite, odinite, orthochamosite, penninite, pannantite, rhipidolite, prochlore, sudoite, thuringite, kaolinite, dickite, and/or nacrite.

The composite film may be obtained by a sulfonation reaction of a sultone compound and an inorganic material having a nano-sized interlayer distance.

The sultone compound may include, e.g., 1,3-propane sultone A, below, 1,4-butane sultone B, below, and/or a compound represented by the Formulae C through S, below.

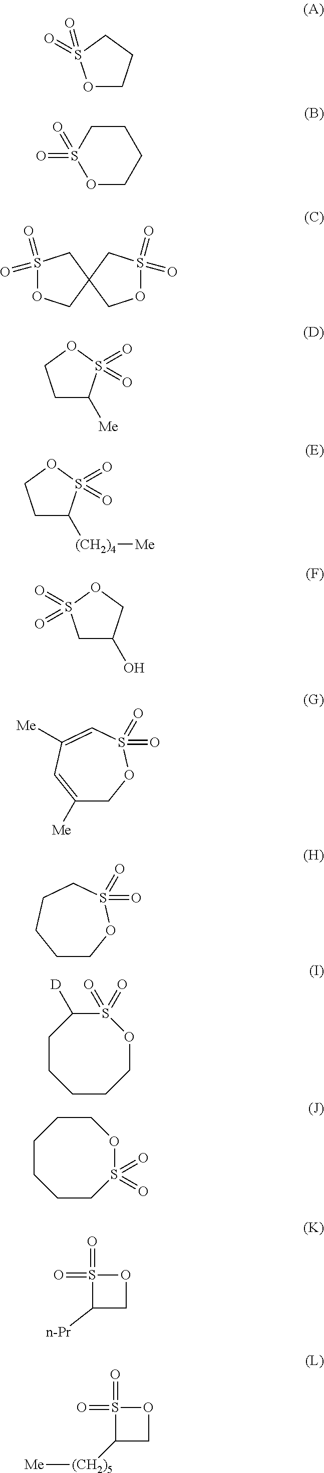

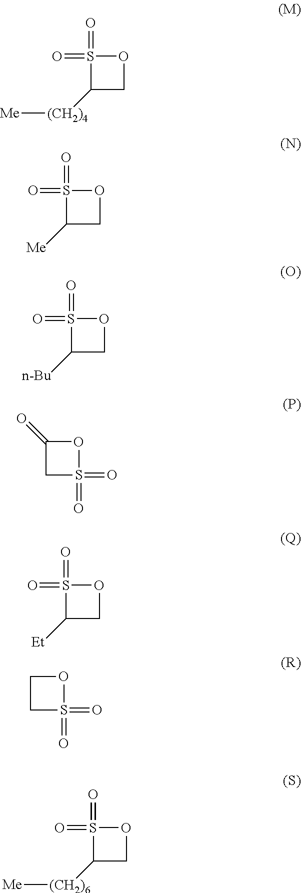

In another implementation, the sultone compound may include, e.g., 1-trifluoromethyl-1,2,2-trifluoroethane sulfonic acid sultone (A′), below, 1-trifluoromethyl-2,2-difluoroethane sulfonic acid sultone (B′), below, 4H-perfluorobutyl-1,2,2-trifluoroethane sulfonic acid sultone (C′), below, a compound represented by Formulae (D′) through (Z′), below, and/or a compound represented by Formulae (a′) through (b′), below.)

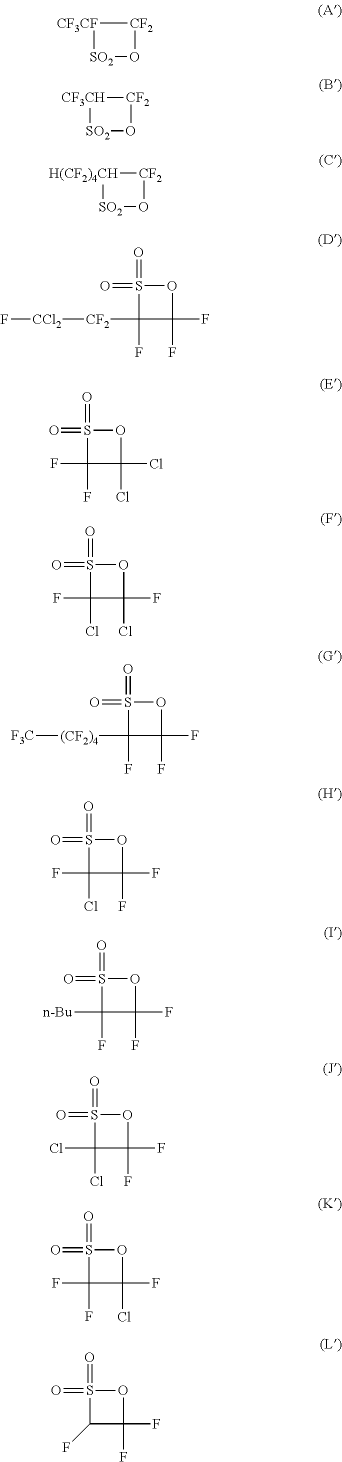

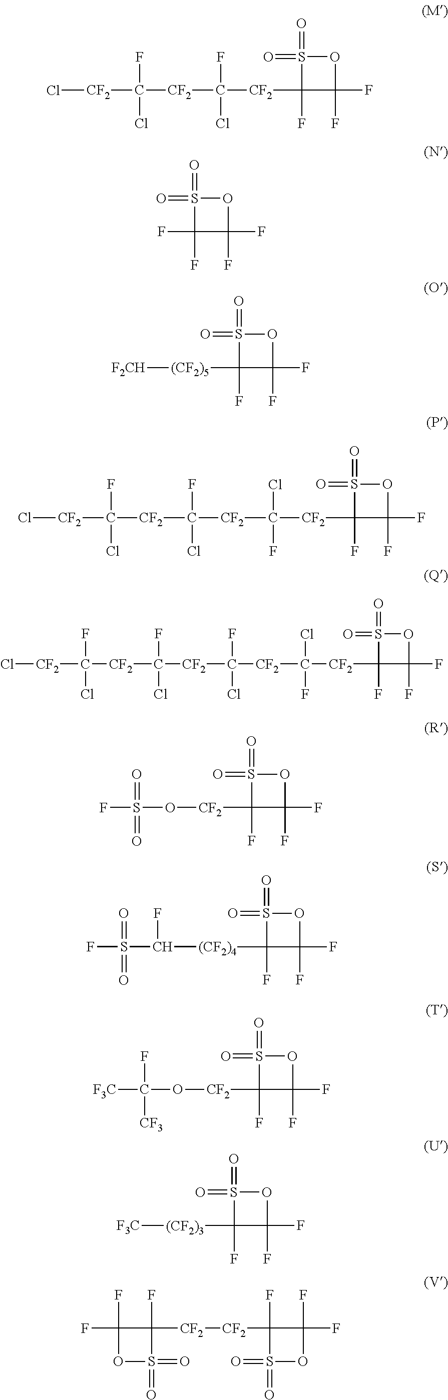

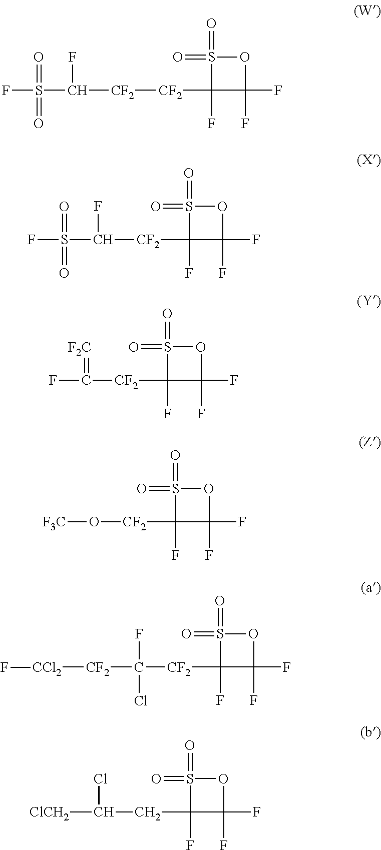

In an implementation, the sultone compound may be, e.g., 1,3-propane sultone, 1,4-butane sultone, or 1,2,2-trifluoro-2-hydroxy-1-trifluoromethylene ethane sulfonic acid sultone.

A sulfonated moisture adsorbate may be obtained by dispersing an inorganic material, e.g., montmorillonite (MMT), in toluene by 10 weight % and reacting it for 24 hours. The sulfonation reaction may be performed for about 6 to about 24 hours at a boiling point temperature of the solvent.

As a result of the sulfonation reaction between the inorganic material having the nano-sized interlayer distance and the sultone compound, a composite in which a sulfonic acid group containing moiety is directly connected to one surface of the inorganic material by an ether linkage may be obtained.

According to an embodiment, the composite film may further include a polymer film.

The polymer film may be one generally used in manufacturing a flexible substrate. In an implementation, the polymer film may include, e.g., polyimide, polyester, polycarbonate, and/or polyethylene terephthalate.

The composite film may include the composite in an amount of about 0.1 parts by weight to about 10 parts by weight based on 100 parts by weight of the polymer film. Maintaining the amount of the composite at about 0.1 parts by weight or greater may help ensure that the composite film is able to be used as a moisture vapor transmission prevention film due to a sufficiently low moisture vapor transmission rate. Accordingly, maintaining the amount of the composite at about 0.1 parts by weight or greater and about 10 parts by weight or less may help ensure that a film having a desirably low moisture vapor transmission rate may be obtained.

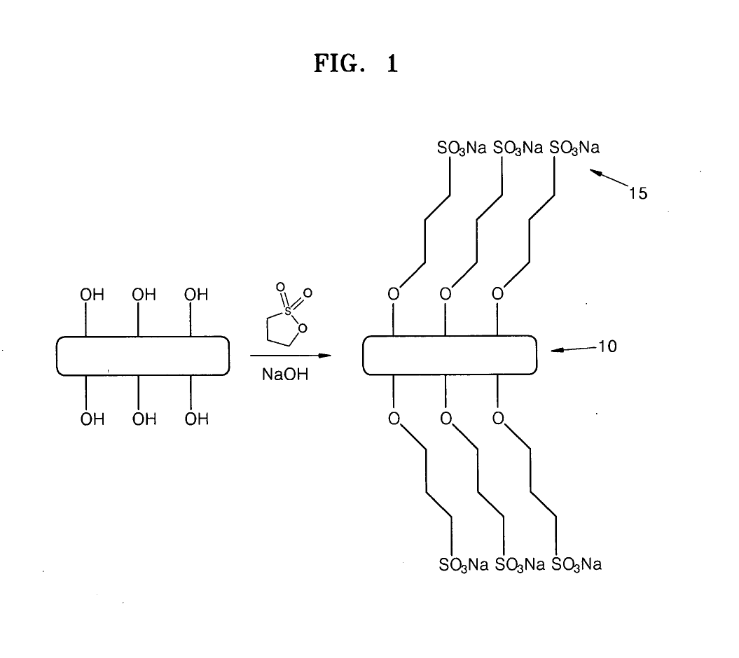

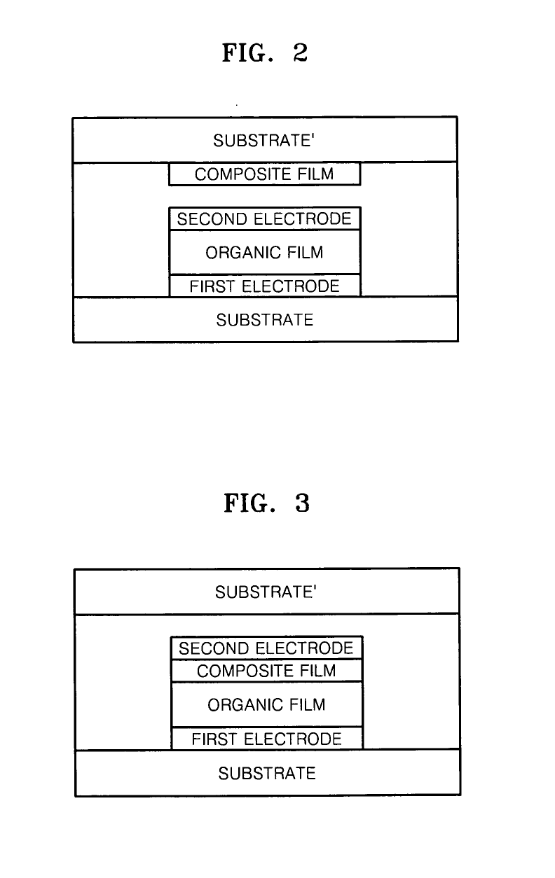

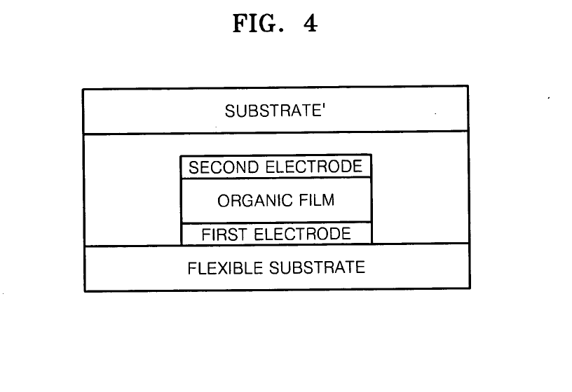

FIG. 1 illustrates a process of manufacturing a composite in which an inorganic material 10 having a nano-sized interlayer distance is directly connected to a sulfonic acid group containing moiety 15 by an ether linkage according to an embodiment. In an implementation, montmorillonite may be used as the inorganic material having a nano-sized interlayer distance and 1,3-propane sultone may be used as the sultone compound. For example, montmorillonite may be reacted with 1,3-propane sultone using a sodium hydroxide composite solution.

In particular, montmorillonite may firstly be dispersed in an acidic aqueous solution. Then, a hydrophilic process may be performed on a surface of the montmorillonite at about 90° C. to about 100° C. for about 6 hours to about 24 hours. Thus, inorganic positive ions, e.g., Na⁺, K⁺, or Mg⁺, may be substituted with H⁺. The acidic aqueous solution may include, e.g., sulfuric acid, hydrochloric acid, or nitric acid, and an amount of the solvent may be about 1000 parts by weight to about 2000 parts by weight, based on 100 parts by weight of the inorganic material. Then, the reacted montmorillonite described above may be reacted with the sultone using the sodium hydroxide composite solution.

Accordingly, a composite, in which montmorillonite having a nano-sized interlayer distance is connected to sulfonic acid group containing moiety (—SO₃Na) by an ether linkage, may be obtained.

According to another embodiment, a flexible substrate including the composite film is provided.

According to the present embodiment, the flexible substrate may include a composite film including, e.g., polyimide and the composite in which montmorillonite having a nano-sized interlayer distance is connected to sulfonic acid group containing moiety (—SO₃Na) by an ether linkage. The sulfonic acid group containing moiety may be, e.g., —(CH₂)₃SO₃Na.

In an implementation, the composite may be thoroughly mixed in a polymer solution and then heat treated to prepare a mixture. The mixture may then be stirred for, e.g., about 3 days, to prepare a film. Then, the film may be thermally treated to prepare the composite film.

According to another embodiment, there is provided an organic light emitting device including a substrate; a first electrode; a second electrode; an organic film interposed between the first electrode and the second electrode; and the composite film according to an embodiment.

The first electrode, the organic film, and the second electrode may be formed on the substrate in the organic light emitting device. For example, the organic light emitting device according to an embodiment may include the first and second electrodes on the substrate and may further include at least one organic layer including, e.g., a hole injection layer (HIL), a hole transport layer (HTL), a hole blocking layer (HBL), an electron transport layer (ETL), and/or an electron injection layer (EIL), between the first electrode and the second electrode. In an implementation, the organic light emitting device may include, e.g., a first electrode/HIL/emission layer (EML)/ETL/EIL/second electrode structure, a first electrode/HIL/HTL/EML/ETL/EIL/second electrode structure, or a first electrode/HIL/HTL/EML/HBL/ETL/EIL/second electrode structure. The HIL may be omitted.

The substrate may be formed of, e.g., glass or transparent plastic having excellent mechanical strength, thermal stability, transparency, surface smoothness, use, and water impermeability.

According to an embodiment, the composite film obtained as described above may be used as the substrate.

The first electrode may be formed on the substrate by using a material for forming a first electrode having a high work function by, e.g., deposition or sputtering. The first electrode may include, e.g., indium-doped thin oxide (ITO), indium zinc oxide (IZO), tin oxide (SnO₂), zinc oxide (ZnO), aluminum (Al), silver (Ag), and/or magnesium (Mg). The first electrode may be formed as a transparent electrode or a reflective electrode.

Then, the HIL may be formed on the first electrode using, e.g., vacuum deposition, spin coating, casting, or Langmuir-Blodgett (LB). The HIL may be formed from, e.g., a phthalocyanine compound such as copper phthalocyanine, a starburst-type amine derivative such as TCTA, m-MTDATA, or m-MTDAPB, or conductive polymer having solubility such as polyaniline/dodecylbenzenesulfonic acid (Pani/DBSA), poly(3,4-ethylenedioxythiophene)/poly(4-styrenesulfonate) (PEDOT/PSS), polyaniline/camphor sulfonic acid (Pani/CSA), and/or polyaniline/poly(4-styrenesulfonate) (PANI/PSS).

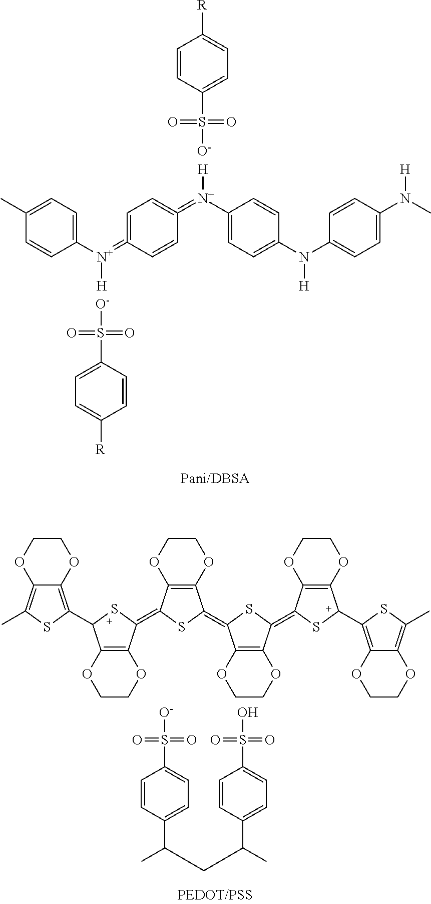

Then, the HTL may be formed on the HIL by using, e.g., vacuum deposition, spin coating, casting, or LB. If the HTL is formed by using vacuum deposition or spin coating, a condition for deposition or coating may vary according to a material used. However, a condition that is nearly the same as that for forming the HIL may generally be selected.

The HTL may be formed from, e.g., a carbazole derivative such as N-phenyl carbazole or polyvinyl carbazole or a general amine derivative having an aromatic fused ring such as N,N′-bis(3-methylphenyl)-N,N-diphenyl-[1,1-biphenyl]-4,4′-diamine (TPD) or N,N′-di(naphthalen-1-yl)-N,N′-diphenyl benzidine (α-NPD).

The EML may be formed on the HTL by using, e.g., vacuum deposition, spin coating, casting, or LB. If the EML is formed by using vacuum deposition or spin coating, a condition for deposition or coating may vary according to a material used. However, a condition that is nearly the same as that for forming the HIL may generally be selected.

The EML may be formed of an appropriate host material and a dopant. The host material may include, e.g., Alq3, (4,4′-N,N′-dicarbazole-biphenyl) (CBP), poly(n-vinylcarbazole) (PVK), and/or 9,10-di(naphthalen-2-yl)anthracene (ADN).

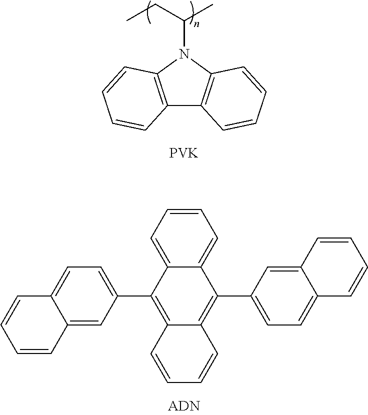

A red dopant may include, e.g., PtOEP, Ir(piq)₃, Btp2Ir(acac), and/or DCJTB.

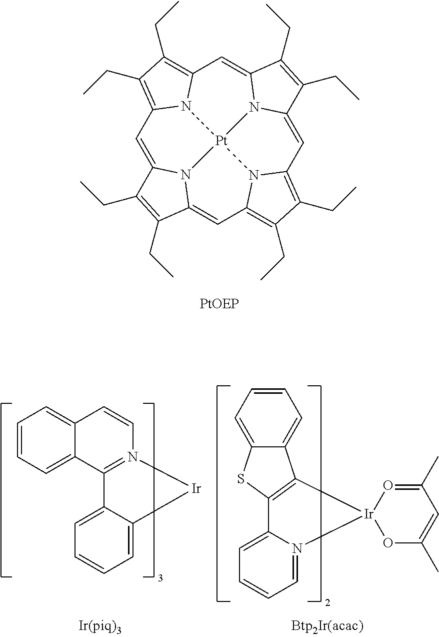

A green dopant may include, e.g., Ir(ppy)₃ (ppy=phenylpyridine), Ir(ppy)₂(acac), Ir(mpyp)₃, and/or C545T.

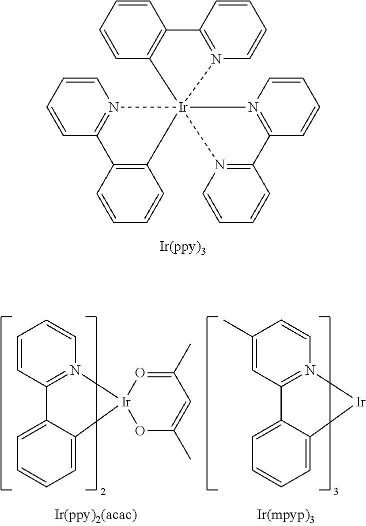

A blue dopant may include, e.g., F₂Irpic, (F₂ppy)₂Ir(tmd), Ir(dfppz)₃, and/or ter-fluorene.

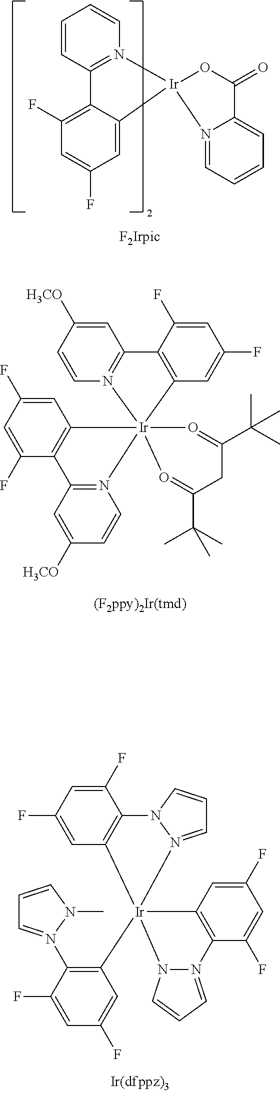

If a phosphorescent dopant is used in the EML, the HBL may be formed between the ETL and the EML by using, e.g., vacuum deposition, spin coating, casting, or LB, in order to, e.g., prevent triplet exciton or hole from being diffused to the ETL. If the HBL is formed by using vacuum deposition or spin coating, a condition for deposition or coating may vary according to a material used. However, a condition that is nearly the same as that for forming the HIL may generally be selected. The HBL may be formed from, e.g., an oxadiazole derivative, triazole derivative, a phenanthroline derivative, BCP, or a material for blocking holes as disclosed in JP 11-329734.

Then, the ETL may be formed by using, e.g., vacuum deposition, spin coating, or casting. If the HTL is formed by using vacuum deposition or spin coating, a condition for deposition or coating may vary according to a material used. However, a condition that is nearly the same as that for forming the HIL may generally be selected. The ETL may be formed from a material that allows electrons injected from a cathode to stably transport and may include, e.g., a well-known electron transport material, a quinoline derivative, in particular, tris(8-quinolinolate)aluminum (Alq3), TAZ, or Balq.

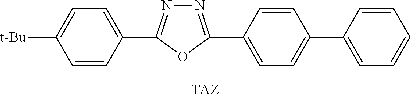

Also, the EIL, which may allow electrons to be easily injected from the cathode, may be formed on the ETL. The EIL may be formed from, e.g., LiF, NaCl, CsF, Li₂O, or BaO, but is not limited thereto. A deposition condition for the EIL may vary according to a material used. However, a condition that is nearly the same as that for forming the HIL may generally be selected.

The second electrode may be formed on the EIL by using, e.g., vacuum deposition or sputtering. The second electrode may be a cathode. A metal for forming the second electrode may include, e.g., a metal having a low work function, a metal alloy, an electrically conductive compound, and/or a mixture thereof. In an implementation, the metal for forming the second electrode may include, e.g., lithium (Li), magnesium (Mg), aluminum (Al), aluminum-lithium (Al—Li), calcium (Ca), magnesium-indium (Mg—In), and/or magnesium-silver (Mg—Ag). Also, in order to obtain a top emission device, a transmissive cathode including, e.g., ITO and/or IZO, may be used.

According to an embodiment, the composite film obtained as above may be formed on the second electrode as an encapsulation film.

A manufacturing method according to an embodiment will now be described with reference to the following examples. These examples are presented for illustrative purposes only and are not intended to limit the scope of the embodiments.

### Example 1

500 ml of 1N sulfuric acid solution and 20 g of montmorillonite were subjected to a reaction at 60° C. for about 4 hours and were sufficiently washed using water.

1300 mmol of toluene was added to a 500 ml round-bottom flask and nitrogen (N₂) was purged to the flask. Then, 60 mmol (6.12 g) of the pre-processed montmorillonite (above) was added to the flask and was stirred to prepare a mixture. Next, 30 mmol (3.66 g) of 1,3-propane sultone and a sodium hydroxide composite solution were added to the mixture to prepare a first reacted mixture. The first reacted mixture was mixed at 110° C. for about 24 hours, cooled, filtered, washed using toluene, and dried in a vacuum oven at 100° C. so as to prepare a composite. 0.090 g of the composite and 18.00 g of a 5 weight % polyimide solution were well mixed, heated at 90° C., and strongly stirred at a speed of 900 rpm so as to prepare a second reacted mixture. Then, the second reacted mixture was stirred for about 3 days to prepare a film. The film was thermally treated in an oven at 130° C. for about 4 hours and at 170° C. for about 3 hours, thereby preparing a composite film.

### Example 2

500 ml of 1N sulfuric acid solution and 20 g of montmorillonite were subjected to a reaction at 60° C. for about 4 hours and were sufficiently washed using water.

1300 mmol of toluene was added to a 500 ml round-bottom flask and nitrogen (N₂) was purged to the flask. Then, 60 mmol (6.12 g) of the pre-processed montmorillonite (above) was added to the flask and was stirred to prepare a mixture. Next, 30 mmol (4.08 g) of 1,4-butane sultone and a sodium hydroxide composite solution were added to the mixture to prepare a first reacted mixture. The first reacted mixture was mixed at 110° C. for about 24 hours, cooled, filtered, washed using toluene, and dried in a vacuum oven at 100° C. so as to prepare a composite.

0.182 g of the composite and 18.00 g of a 5 weight % polyimide solution were well mixed, put in an autoclave container, and subjected to a reaction for 24 hours at 90° C. in 80 psi so as to prepare a reacted film. After the reaction was completed, the reacted film was thermally treated in an oven at 130° C. for about 4 hours and at 170° C. for about 3 hours, thereby preparing a composite film.

### Example 3

500 ml of 1N sulfuric acid solution and 20 g of montmorillonite were subjected to a reaction at 60° C. for about 4 hours and were sufficiently washed using water.

32 ml of toluene was added to a 100 ml round-bottom flask and nitrogen (N₂) was purged to the flask. Then, 20 mmol (2.04 g) of the pre-processed montmorillonite (above) was added to the flask and was stirred to prepare a mixture. Next, 30 mmol (2.42 g) of 1-trifluoromethyl-1,2,2-trifluoroethane sulfonic acid sultone and a sodium hydroxide composite solution were added to the mixture to prepare a first reacted mixture. The first reacted mixture was mixed at 110° C. for about 24 hours, cooled, filtered, washed using toluene, and dried at room temperature so as to prepare a composite.

0.090 g of the composite and 18.00 g of a 5 weight % polyimide solution were well mixed, heated at 90° C., and strongly stirred at a speed of 900 rpm so as to prepare a second reacted mixture. Then, the second reacted mixture was stirred for about 3 days to prepare a film and the film was thermally treated in an oven at 130° C. for about 4 hours and at 170° C. for about 3 hours, thereby preparing a composite film.

### Comparative Example 1

18.00 g of a polyimide solution including 5 weight % of montmorillonite (MMT) was used to prepare a polymer film. The film was thermally treated in an oven at 130° C. for about 4 hours and at 170° C. for about 3 hours, thereby preparing a composite film.

Characteristics of the composite films prepared according to Examples 1 through 3 and Comparative Example 1 were evaluated as follows.

Permeability of water and methanol was measured for the composite films prepared according to Examples 1 through 3 and Comparative Example 1 and the results are shown in Table 1.

TABLE 1

| Substrate | WVTR (g/m²/day) |
|---|---|
| Example 1 | 1.0 |
| Example 2 | 0.3 |
| Example 3 | 0.8 |
| Comparative Example 1 | 8.0 |

Referring to Table 1, the composite films of Examples 1 through 3 exhibited lower permeability than the composite film of Comparative Example 1. The permeabilities of the composite films of Examples 1 through 3 and Comparative Example 1 were 1.0, 0.3, 0.8, and 8.0 g/m²/day, respectively.

The composite film according to an embodiment may provide excellent barrier properties against oxygen or moisture. Thus, a lifespan of the flexible substrate and organic light emitting device including the composite film may be improved. Also, a method of manufacturing the composite film according to an embodiment may be simple.

A substrate formed of a polymer film including inorganic materials may have low CTE and high thermal stability (i.e., high Tg) and thus may have a high barrier property against gas and moisture.

Exemplary embodiments have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A composite film, comprising:
a composite including a sulfonic acid group containing moiety connected by an ether linkage (—O—) to an inorganic material having a nano-sized interlayer distance.

2. The composite film as claimed in claim 1, wherein the sulfonic acid group containing moiety includes —(CH₂)ₙSO₃M, wherein M is Na, K, Ca, or Ba and n is and integer of about 1 to about 13.

3. The composite film as claimed in claim 1, wherein the sulfonic acid group containing moiety includes —C(R₂)(X)CF₂SO₃M, wherein M is Na, K, Ca, or Ba, R₂ is —F, —CF₃, —SF₅, ═SF₄, —SF₄Cl, —CF₂CF₃, or —H(CF₂)₄, and X is —F, —H, —Cl, or —CF₃.

4. The composite film as claimed in claim 1, wherein the inorganic material includes at least one of montmorillonite, koline, hydrated sodium calcium aluminum magnesium silicate hydroxide, pyrophyllite, talc, vermiculite, sauconite, saponite, nontronite, amesite, baileychlore, chamosite, clinochlore, kaemmererite, cookeite, corundophilite, daphnite, delessite, gonyerite, nimite, odinite, orthochamosite, penninite, pannantite, rhipidolite, prochlore, sudoite, thuringite, kaolinite, dickite, and nacrite.

5. The composite film as claimed in claim 1, wherein the composite is obtained by sulfonation of the inorganic material by a sultone compound.

6. The composite film as claimed in claim 5, wherein the sultone compound includes at least one of 1,3-propane sultone, 1,4-butane sultone, and 1-trifluoromethyl-1,2,2-trifluoroethane sulfonic acid sultone.

7. The composite film as claimed in claim 1, further comprising a polymer.

8. The composite film as claimed in claim 7, wherein the polymer includes at least one of polyimide, polyester, polycarbonate, and polyethylene terephthalate.

9. The composite film as claimed in claim 8, wherein the composite is included in an amount of about 0.1 parts by weight to about 10 parts by weight based on 100 parts by weight of the polymer.

10. A flexible substrate, comprising a composite film including a composite including a sulfonic acid group containing moiety connected by an ether linkage (—O—) to an inorganic material having, a nano-sized interlayer distance.

11. The flexible substrate as claimed in claim 10, wherein the composite film further includes polyimide.

12. The flexible substrate as claimed in claim 11, wherein the sulfonic acid group containing moiety is —(CH₂)₃SO₃Na.

13. An organic light emitting device, comprising:
a first electrode, a second electrode, and an organic film interposed between the first electrode and the second electrode; and
a composite film including a sulfonic acid group containing moiety connected by an ether linkage (—O—) to an inorganic material having a nano-sized interlayer distance.

14. The device as claimed in claim 13, wherein the composite film is a flexible substrate.

15. The device as claimed in claim 13, wherein the composite film is an encapsulation film on the second electrode.
